(19) United States
(12) Patent Application Publication
(10) Pub. No.: US 2019/0321308 A1
(43) Pub. Date: Oct. 24, 2019

(54) COMPOSITIONS CONTAINING TETRAHYDROCURCUMINOIDS FOR IMPROVEMENT OF ORAL HEALTH AND HYGIENE

(71) Applicants: Muhammed Majeed, Edison, NJ (US); Kalyanam Nagabhushanam, East Windsor, NJ (US); Shaheen Majeed, Springville, UT (US)

(72) Inventors: Muhammed Majeed, Edison, NJ (US); Kalyanam Nagabhushanam, East Windsor, NJ (US); Shaheen Majeed, Springville, UT (US)

(21) Appl. No.: 15/955,905

(22) Filed: Apr. 18, 2018

(51) Int. Cl. A61K 31/12; A61K 8/35; A61Q 11/00; A61K 9/20; A61P 1/02; A61K 9/48

(52) U.S. Cl. CPC A61K 31/12; A61K 8/35; A61K 9/48; A61K 9/20; A61P 1/02; A61Q 11/00

(57) ABSTRACT

Disclosed, is a novel composition containing tetrahydrocurcuminoids for the improvement of oral, health and hygiene. More specifically, disclosed is a novel composition of tetrahydrocurcuminoids and its application in the therapeutic management of canker sore and gingivitis.

### FIELD OF THE INVENTION

The present invention relates to a novel composition containing tetrahydrocurcuminoids for the improvement of oral health and hygiene. More specifically, the invention relates to a novel tetrahydrocurcuminoid composition and its application in therapeutic management of canker sore and gingivitis.

### BACKGROUND OF THE INVENTION

### Description of Prior Art

The health and hygiene of the teeth, the mouth, and mucosal tracts of the oral cavity is important to a person's overall health. The common type of oral disease include dental cavities, caries, bad breath and gum diseases, including canker sore, gingivitis, and periodontitis. Canker sores and gingivitis are the most commonly occurring oral problems. Recurrent aphthous stomatitis (RAS) (aphthae; canker sores) is one of the most common, painful oral ailments. characterized by multiple, small, round, or ovoid ulcers with circumscribed margins, erythematous haloes, and yellow or grey floors that presents itself in childhood or adolescence. (Scully C, Porter S. Oral mucosal disease: recurrent aphthous stomatitis, Br J Oral Maxillofac Surg. 2008 April; 46(3):198-206; Preeti L, et al., Recurrent aphthous stomatitis, J Oral Maxillofac Pathol., 2011; 15:252-6). Gingivitis affects an estimated 80% of the population and is the world's most predominant inflammatory periodontal disease. The disease is initiated with a pathogenic biofilm, or plaque, around the teeth followed by a host immune-inflammatory response that advances the disease.

As a main stay to maintain oral hygiene, mechanical plaque control measures are generally employed. But, these techniques are either time consuming or require motivation and skill to be performed well for effective relief against the symptoms. Hence, antimicrobial agents such as chlorhexidine, metronidazole etc are being used extensively as an adjunct to mechanical cleaning. However, these artificial drugs have unpleasant side-effects and hence a safe and effective natural/herbal source in providing relief against oral health problems is the current industrial requirement.

Turmeric, especially curcumin is an effective natural alternative for the management of oral health problems which is specifically documented in the following:
1. Clinical efficacy of turmeric use in gingivitis: A comprehensive review, Stoyell K A, Mappus J L, Gandhi M A, Complement Ther Clip Pract. 2016 November; 25: 13-17).
2. Efficacy of curcumin in the treatment of chronic gingivitis: a pilot study. Muglikar, S., Patil, K. C., Shivswami, S., and Hegde, R., Oral Health and Preventive Dentistry. 2012; 11(1):81-86)
3. comparison of effectiveness of curcumin with triamcinolone acetonide in the gel form in treatment of minor recurrent aphthous stomatitis: A randomized clinical trial., Deshmukh R A, Bagewadi A S, Int J Pharm Investig. 2014 July; 4(3):138-41).
4. Oral care compositions, Brading et al., U.S. Pat. No. 8,916,139

Owing to the problems associated with curcumin when incorporated in oral care compositions like bioavailability, degradation, discoloration, there is a need to develop a stable natural alternative for management of oral health and hygiene, especially for canker sore and gingivitis because of their different pathological features. The invention solves the above problem by disclosing a composition containing tetrahydrocurcuminoids for the therapeutic management of canker sore and gingivitis.

The principle objective of the invention is to disclose a composition containing tetrahydrocurcuminoids and for the improvement of oral health and hygiene.

It is another objective of the invention to disclose a composition containing tetrahydrocurcuminoids for therapeutic management of canker sore and gingivitis.

### SUMMARY OF THE INVENTION

The present invention discloses a composition containing tetrahydrocurcuminoids for the improvement of oral health and hygiene. Specifically the invention discloses a novel composition of tetrahydrocurcuminoids for the therapeutic management of canker sore and gingivitis.

### BRIEF DESCRIPTION OF THE FIGURES (FIGS. 1a, 1b, 1c, 2a AND 2b)

FIG. 1a shows graphical representation of median VAS score at three time points; p value <0.001 using Friedman ANNOVA,

FIG. 1b shows graphical representation of median throat relief score at three points

FIG. 1c shows graphical representation of distribution of patients as per throat numbness at different time points

FIGS. 2a and 2b shows Line plots mean values of different LOE & SILNESS Index parameters and total points at three times in Gingivitis group

### DESCRIPTION OF MOST PREFERRED EMBODIMENTS

In a most preferred embodiment, the invention relates to a novel composition containing tetrahydrocurcuminoids for the improvement of oral health and hygiene. In a related embodiment the composition contains tetrahydrocurcumin, tetrahydrodemethoxycurcumin, tetrahydrobis-demethoxycurcumin, octahydrocurcuminoids and hexahydrocurcuminoids. In a related embodiment the composition is formulated with pharmaceutically, nutraceutically and cosmeceutically acceptable excipients, adjuvants, diluents or carriers and administered in the form of tablets, capsules, syrups, gummies, powders, suspensions, emulsions, chewables, candies, creams, gels, lotions, ointments, mouth wash, tooth paste and eatables. In a related embodiment, the tablets, capsules, syrups, gummies, powders, suspensions, emulsions, chewables, candies, creams, gels, lotions, ointments, mouth wash, tooth paste and eatables, are available separately or together in an oral health kit containing the said forms.

In another preferred embodiment, the present invention discloses a method for therapeutic management of canker sore in mammals, said method comprising steps of administering an effective concentration of a composition containing tetrahydrocurcuminoids to mammals in need of such therapy, to bring about reduction in symptoms associated with canker sore. In another related embodiment, the symptoms of canker sore include, but not limited to, throat pain, ulcer, lesion, throat numbness, sore throat, bleeding, swelling at the site, reddening at the site, inflammation at the site, burning, swelling and paid in lymph node, difficulty in chewing and difficulty in swallowing. In a yet another related embodiment the composition is formulated with pharmaceutically, nutraceutically and cosmeceutically acceptable excipients, adjuvants, diluents or carriers and administered in the form of tablets, capsules, syrups, gummies, powders, suspensions, emulsions, chewables, candies, creams, gels, lotions, ointments, mouth wash, tooth paste and eatables. In a related embodiment, the tablets, capsules, syrups, gummies, powders, suspensions, emulsions, chewables, candies, creams, gels, lotions, ointments, mouth wash, tooth paste and eatables, are available separately or together in an oral health kit containing the said forms. In another embodiment, the mammal is preferably human.

In another preferred embodiment, the present invention discloses a method for therapeutic management of gingivitis in mammals, said method comprising steps of administering an effective concentration of a composition containing tetrahydrocurcuminoids to mammals in need of such therapy, to bring about reduction in symptoms associated with gingivitis. In another related embodiment, the symptoms of gingivitis include, but not limited to, taste acceptability, burning sensation, dryness, soreness, ulcer, staining of teeth and tongue, and allergy. In a yet another related embodiment the composition is formulated with pharmaceutically, nutraceutically and cosmeceutically acceptable excipients, adjuvants, diluents or carriers and administered in the form of tablets, capsules, syrups, gummies, powders, suspensions, emulsions, chewables, candies, creams, gels, lotions, ointments, mouth wash, tooth paste and eatables. In a related embodiment, the tablets, capsules, syrups, gummies, powders, suspensions, emulsions, chewables, candies, creams, gels, lotions, ointments, mouth wash, tooth paste and eatables, are available separately or together in an oral health kit containing the said forms. In another embodiment, the mammal is preferably human.

Specific illustrative examples enunciating the most preferred embodiments are included herein below.

### EXAMPLES

### Example 1: Experimental Procedures

Product Description

The study supplements were supplied in the form of chewable tablets with oral dosing twice daily containing 100 mg of tetrahydrocurcuminoids.

Ethics and Informed Consent

This trial was conducted in accordance with the clinical research guidelines established by the Drugs and Cosmetics Act, 1940 of India, Drugs and Cosmetics Rules, 1945 of India, Ethical Guidelines for Biomedical Research on Human Participants, 2006 of Indian Council of Medical Research (ICMR) in India, the principles enunciated in the Declaration of Helsinki, Edinburgh (Anonymous, 2000) and the ICH-harmonized tripartite guideline regarding Good Clinical Practice (GCP). The study was approved by the Institutional ethics committee, a written informed consent was obtained, and the study was registered at Clinical Trials Registry-India (www.ctri.nic.in) (Identifier: CTRI/2017/01/007670). There were no changes to the methods or planned endpoints after study initiation.

Participants

Subjects were included in the study if indicated “Yes” to all of the inclusion criteria and “No” to all of the exclusion criteria.

Inclusion Criteria:

1. Male and/or female—Healthy, with no systemic disease (diabetes).
2. Age between 18 to 60 years.
3. Patients having mouth ulcers with throat pain or Patients with mild to moderate gingivitis.
4. Patients having erupted teeth.
5. Willing to come for regular follow-up visits.
6. Able to give written informed consent.

Exclusion Criteria:

### 1. Smokers.

2. Presence of any systemic hematological problems.
3. Drug or alcohol abuse.
4. Under systemic or topical corticosteroid therapy.
5. Pregnant women or lactating mothers
6. History of HIV and other viral infections.
7. History of any antibiotic therapy in past 3 months.
8. History of oral prophylaxis within 6 months previous to the study
9. Patients with mouth breathing habit.
10. Patients using orthodontic and prosthodontics appliances
11. Are on analgesics or antiseptics.
12. Patients who have received any periodontal therapy in the past 6 months.

Trial Design

A Prospective, open label, two arm, clinical study to evaluate the efficacy and safety in the treatment of canker sore and gingivitis was conducted in India at two different sites: 1) Noble Hospital Pvt. Ltd., Pune, Maharashtra, India. 2) Sri Venkateshwara Hospital, Bangalore, India. The sample size of the study was 60, with 30 subjects assigned to each of the two study arms in a manner there would be 30 subjects with canker sore and throat pain and 30 subjects with gingivitis. However, 31 subjects were enrolled for Canker Sore and 29 were enrolled for Gingivitis after obtaining signed Informed Consent from these subjects. Subjects were non blinded as it was an open label study. Compliance with study supplement was reviewed at each visit. The subjects were allowed to take their regular diet. The study consisted of a 21 day intervention period. Subjects visited the study site on screening (day −3 i.e. three days prior to baseline visit), baseline (day 0), visit day 14 and day 21.

Intervention

Male and female subjects between the age group of 18 to 60 years suffering with mouth ulcers along with throat pain were screened for the study. The screening procedures included medical history, medication and treatment history, physical examination, demographics and vital signs, pregnancy test in case of females, laboratory investigation (haematology), and efficacy assessments were conducted. Comorbid conditions including any active, chronic, recurrent or latent infections.

All enrolled subjects were instructed to self administer one chewable tablet (oral dosing) twice daily containing 100 mg of tetrahydrocurcuminoids for a period of 21 days.

Efficacy Outcomes

For Canker sore with throat pain—

Number of ulcers healed.

Pain intensity Assessment by VAS (Visual Analogue Scale).

Ulcer size reduction.

Duration of complete healing of ulcers.

Clinical Photographs of the Lesion.

Throat Numbness Scale.

Sore Throat Relief Scale.

For Gingivitis—

Loe and Silness Gingival index.

Subjective criteria for Gingivitis.

Objective criteria for Gingivitis.

Clinical Photographs of gingival region.

Secondary Objective

To assess the safety of tetrahydrocurcuminoids in the treatment of Canker sore with throat pain and gingivitis.

Safety Outcomes:

Any reports of adverse or serious adverse events.

Abnormal vital signs & lab parameters.

Statistical Analysis of Efficacy Evaluation

The average scores were calculated separately on a per-subject basis at Baseline and Day 21. For statistical purpose the Screening scores were considered as Baseline. The Day 21 means for each endpoint were modeled separately with analysis of covariance, using the respective baseline score as a covariate. All treatment comparisons were tested at the two-sided 5% significance level.

### Example 2: Results

This Study was conducted in India across two sites from November 2016 to March 2017. A total of 60 adult patients were enrolled and treated for 21 days with twice a day dosing every morning and evening. A total of 11 women and 49 men participated in the study. Total of 31 patients were enrolled in canker sore, out of which 1 patient dropped out and total 29 patients were enrolled in gingivitis arm. The female to male ratio completing the study was 10:49 with one female dropout. Patients enrolled were having no other physical abnormalities except either canker sore or gingivitis.

Efficacy Outcome

Canker Sore

The clinical parameter like systolic and diastolic BP, pulse rate, heart rate, respiratory rate and BMI were compared using repeated measure analysis of variance and was insignificant in between two time points. Physical examination revealed that general appearance, heart, lungs, abdomen, extremities and neurological functions were normal.

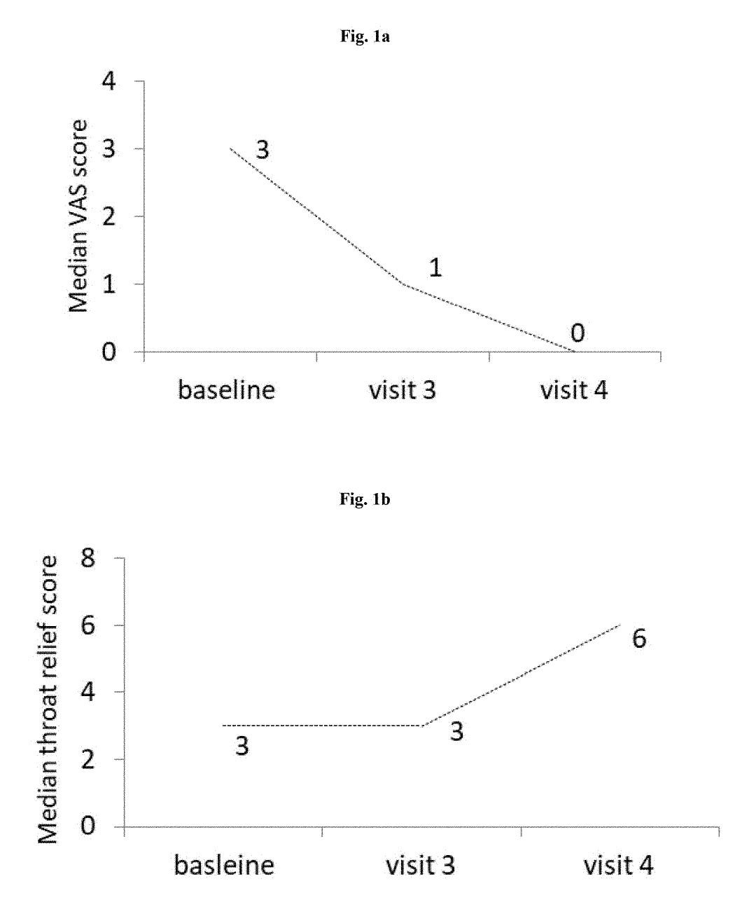

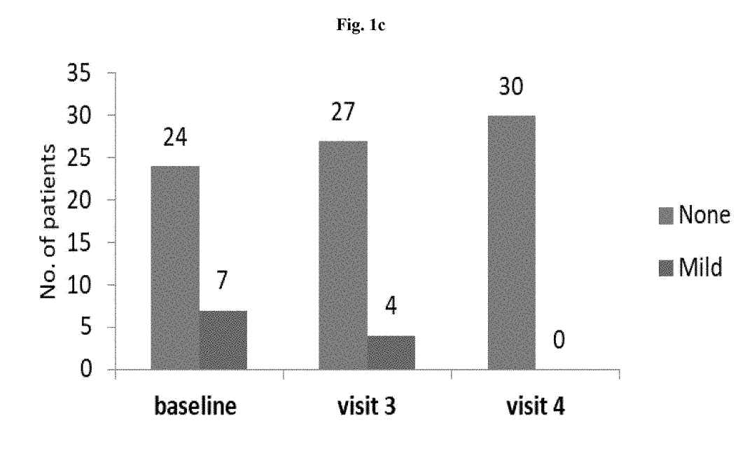

There was gradual decrease in numbers of lesion across the study period (Table 1). The duration taken for tetrahydrocurcuminoids to treat canker sore was an average of 21 days as almost all the symptoms were reduced at the final visit. There was statistically significant reduction in number of cases with numbness after treatment (FIG. 1a). Further, comparison of throat relief score before and after treatment showed significant improvement (FIG. 1b). Also, VAS scores before and after treatment has shown significant reduction from median score of 3 to 0 (FIG. 1c).

TABLE 1
Distribution of patients with different no of lesions across
study visits.

| | No. of patients | | |
|---|---|---|---|
| Visits/no. of lesions | Baseline (n = 31) | Visit 3 (n = 31) | Visit 4 (n = 30) |
| Multiple | 5 | 4 | 0 |
| Single | 26 | 27 | 0 |

There was a significant change in the symptoms of canker sore with throat pain like pain at site, burning at site, reddening, difficulty in chewing and swallowing post treatment. Bleeding was insignificant due to treatment during the study period (Table 2).

TABLE 2
Comparison of signs and symptoms before and after treatment of
canker sore patients

| | No. of patients | | |
|---|---|---|---|
| | Baseline visit | Visit 3 | Visit 4 |
| Signs & symptoms | (n = 31) | (n = 31) | (30) |
| Bleeding | 2 | 0 | 0 |
| Pain at the site | 23 | 13 | 2 |
| Burning at the site | 16 | 2 | 1 |
| Reddening at the site | 21 | 8 | 0 |
| Inflammation at the site | 0 | 0 | 0 |
| Swelling and pain at lymph node | 0 | 0 | 0 |
| Difficulty in chewing | 12 | 5 | 0 |
| Difficulty in swallowing | 18 | 6 | 1 |

P-value < 0.001 except for bleeding (P > 0.05)

Gingivitis

The clinical parameter like systolic and diastolic BP, pulse rate, heart rate, respiratory rate and BMI were compared using analysis of covariance and was insignificant in between two time points. The general appearance, heart, lungs, abdomen, extremities and neurological functions were shown normal by physical examination. The duration taken by tetrahydrocurcuminoids to treat gingivitis is in average of 14-21 days.

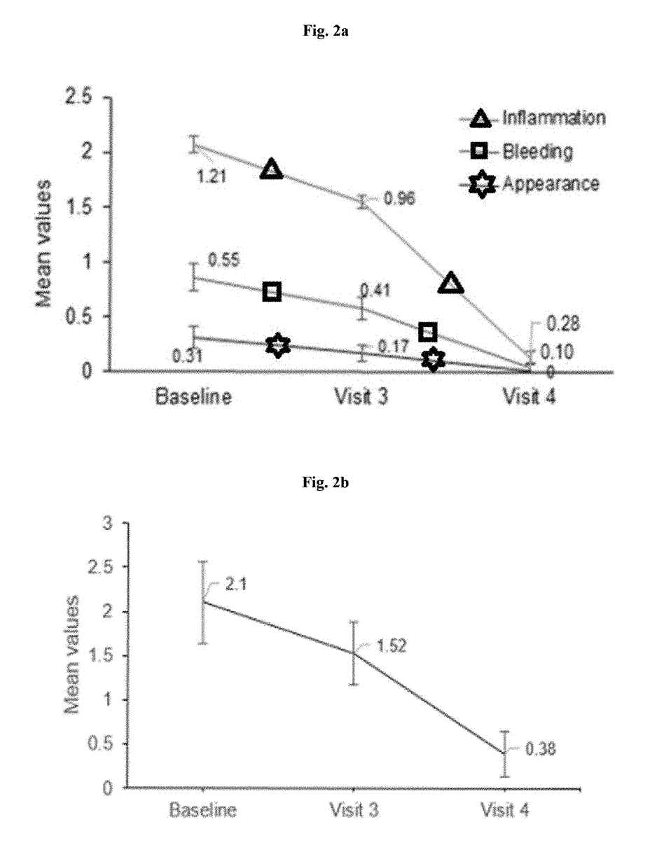

Loe & Silness index was compared between two time points. Appearance differed significantly before and after treatment as indicated by P-value of 0.008 using Wilcoxon signed rank test. Also, there was significant difference in the bleeding scores between baseline and visit 4 as revealed by P-value of 0.002. Further, inflammation was also significantly reduced at visit 4 compared to baseline (P<0.001). Overall, the total points reduced from median of 2 at baseline to 0 at visit 4; and the reduction was statistically significant (P<0.001) (FIG. 2).

The gingivitis Criteria compared between baseline and visit 4 didnt show any statistical significance due to a fairly constant response at visit 3 and visit 4 (Table 3).

TABLE 3
Comparison of gingivitis criteria before and after treatment

| | | No of patients | | |
|---|---|---|---|---|
| GINGIVITIS CRITERIA | | Baseline | Visit 3 | Visit 4 |
| Taste acceptability | Acceptable | 29 | 29 | 29 |
| | Unacceptable | 0 | 0 | 0 |
| Burning sensation | Present | 13 | 2 | 0 |
| | Absent | 16 | 27 | 19 |
| Dryness/Soreness | Present | 1 | 0 | 0 |
| | Absent | 28 | 29 | 29 |
| Ulcer formation | Present | 2 | 0 | 0 |
| | Absent | 27 | 29 | 29 |
| Staining of teeth | Present | 14 | 16 | 11 |
| | Absent | 15 | 13 | 18 |
| Staining of tongue | Present | 1 | 0 | 1 |
| | Absent | 28 | 29 | 28 |
| Allergy | Present | 0 | 0 | 0 |
| | Absent | 29 | 29 | 29 |

*Using McNemar test;
‘—’no statistics obtained as the patient responses was fairly constant during visit 3 and 4.

Safety Results

There were insignificant changes in the parameters like systolic BP, diastolic BP, pulse rate, heart rate, respiratory rate and BMI between two times. There were no abnormal laboratory values reported during the study and no significant change in haematology. There was no change in vital parameters as compared to baseline. None of the patient experienced AEs during the study period in both canker sore and gingivitis arm. Thus, the tetrahydrocurcuminoids composition was acceptable in the study population and demonstrated as a completely safe preparation.

### Example 3: Compositions/Formulations Containing Tetrahydrocurcuminoids

Tables 4-9 provide illustrative examples of nutraceutical formulations containing tetrahydrocurcuminoids for the management of canker sore and gingivitis

TABLE 4
Tetrahydrocurcuminoids Chewable Tablet

Active Ingredients
Tetrahydrocurcuminoids 50.0 mg
Excipients
Sugar, Sugar binders, Flavouring agent, Acidulant
(Citric acid/Tartaric acid/Malic acid), Xylitol,
Magnesium stearate

TABLE 5
Tetrahydrocurcuminoids Chewable Tablet

Active Ingredients
Tetrahydrocurcuminoids 50.0 mg
Multivitamins, Coenzyme Q10, Glutathione, Garlic extract, Vitamin B12,
Folic acid, Vitamin C, Amino acids (Lysine and Glutamine), minerals
Excipients
Sugar, Sugar binders, Flavouring agent, Acidulant (Citric acid/Tartaric
acid/Malic acid), Xylitol, Magnesium stearate

TABLE 6
Tetrahydrocurcuminoids Tablet

Active Ingredients
Tetrahydrocurcuminoids 400 mg
Piperine
Excipients
Povidone (PVPK 30), CrosPovidone, Microcrystalline cellulose,
Colloidal silicon dioxide, Magnesium stearate,
Coating excipients
Hypromellose, Propylene Glycol

TABLE 7
Tetrahydrocurcuminoids Tablet

Active Ingredients
Tetrahydrocurcuminoids 400 mg
Piperine, Multivitamins, Coenzyme Q10, Glutathione, Garlic extract,
Vitamin B12, Folic acid, Vitamin C, Amino acids (Lysine and
Glutamine), minerals
Excipients
Povidone (PVPK 30), CrosPovidone, Microcrystalline cellulose, Colloidal
silicon dioxide, Magnesium stearate,
Coating excipients
Hypromellose, Propylene Glycol

TABLE 8
Tetrahydrocurcuminoids Capsules

Active Ingredients
Tetrahydrocurcuminoids 200 mg
Piperine
Excipients
Microcrystalline cellulose, Colloidal
silicon dioxide, Magnesium stearate

TABLE 9
Tetrahydrocurcuminoids Capsules

Active Ingredients
Tetrahydrocurcuminoids 200 mg
Piperine, Multivitamins, minerals, Coenzyme Q10, Glutathione,
Garlic extract, Vitamin B12, Folic acid, Vitamin C, Amino acids
(Lysine and Glutamine)
Excipients
Microcrystalline cellulose, Colloidal silicon dioxide, Magnesium
stearate

Table 10 and 11 provides illustrative examples for a mouth gel and mouth wash formulation containing tetrahydrocurcuminoids for the management of canker sore and gingivitis

TABLE 10
Tetrahydrocurcuminoids mouth gel

Active Ingredients
Tetrahydrocurcuminoids 20-200 mg
Triphala Extract
Excipients
Disodium EDTA, Glycerin, Biopol 10, Cremophor RH 40, Menthol,
Zemea (Propanediol), Sodium Benzoate, Potassium Sorbate,
Sodium Hydroxide, Sodium Saccharin

TABLE 11
Tetrahydrocurcuminoids mouth wash

Active Ingredients
Tetrahydrocurcuminoids 20-200 mg
Methyl salicylate, Fluoride, Cetylpyridinium chloride
Excipients
Sucralose, Sodium Saccharin, Flavouring agent, polysorbate.

Table 12 provides an illustrative example for a toothpaste formulation containing tetrahydrocurcuminoids for the management of canker sore and gingivitis

TABLE 12
Tetrahydrocurcuminoids tooth paste

Active Ingredients
Tetrahydrocurcuminoids 20-200 mg
Fluoride
Excipients
Titanium dioxide, Xylitol, Flavouring agents,
Preservatives, Sodium saccharin, citroxain

Oral Health Kit

The invention also provides an illustrative example of an oral health kit comprising of formulation containing tetrahydrocurcuminoids which include, but not limited to, the following:

1. Chewable tablets, candies and gummies

2. Tablets and capsules containing tetrahydrocurcuminoids

3. Creams, lotions, ointments, gels containing tetrahydrocurcuminoids

4. Mouth wash and tooth paste

The above formulations are just illustrative examples, any formulation containing the above active ingredient intended for the said purpose will be considered equivalent.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

We claim:

1. A composition containing tetrahydrocurcuminoids for the improvement of oral health and hygiene.

2. The composition as in claim 1, wherein the composition is formulated with pharmaceutically, nutraceutically and cosmeceutically acceptable excipients, adjuvants, diluents or carriers and administered in the form of tablets, capsules, syrups, gummies, powders, suspensions, emulsions, chewables, candies, creams, gels, lotions, ointments, mouth wash, tooth paste and eatables.

3. The formulation as in claim 2, wherein the tablets, capsules, syrups, gummies, powders, suspensions, emulsions, chewables, candies, creams, gels, lotions, ointments, mouth wash tooth paste and eatables, are available separately or together in an oral health kit containing the said forms.

4. A method for therapeutic management of canker sore in mammals, said method comprising steps of administering an effective concentration of a composition containing tetrahydrocurcuminoids to mammals in need of such therapy, to bring about reduction in symptoms associated with canker sore.

5. The method as in claim 4, wherein the symptoms of canker sore include, but not limited to, throat pain, ulcer, lesion, throat numbness, sore throat, bleeding, swelling at the site, reddening at the site, inflammation at the site, burning, swelling and pain in lymph node, difficulty in chewing and difficulty in swallowing.

6. The method as in claim 4, wherein the composition is formulated with pharmaceutically, nutraceutically and cosmeceutically acceptable excipients, adjuvants, diluents or carriers and administered in the form of tablets, capsules, syrups, gummies, powders, suspensions, emulsions, chewables, candies, creams, gels, lotions, ointments, mouth wash, tooth paste and eatables.

7. The method as in claim 6, wherein the formulation in the form of tablets, capsules, syrups, gummies, powders, suspensions, emulsions, chewables, candies, creams, gels, lotions, ointments, mouth wash tooth paste and eatables, are available separately or together in an oral health kit containing the said forms.

8. The method as in claim 4, wherein the mammal is preferably human.

9. A method for therapeutic management of gingivitis in mammals, said method comprising steps of administering an effective concentration of a composition containing tetrahydrocurcuminoids to mammals in need of such therapy, to bring about reduction in symptoms associated with gingivitis.

10. The method as in claim 9, wherein the symptoms of gingivitis include, but not limited to, taste acceptability, burning sensation, dryness, soreness, ulcer, staining of teeth and tongue, and allergy.

11. The method as in claim 9, wherein the composition is formulated with pharmaceutically, nutraceutically and cosmeceutically acceptable excipients, adjuvants, diluents or carriers and administered in the form of tablets, capsules, syrups, gummies, powders, suspensions, emulsions, chewables, candies, creams, gels, lotions, ointments, mouth wash, tooth paste and eatables.

12. The method as in claim 11, wherein the formulation in the form of tablets, capsules, syrups, gummies, powders, suspensions, emulsions, chewables, candies, creams, gels, lotions, ointments, mouth wash tooth paste and eatables, are available separately or together in an oral health kit containing the said forms.

13. The method as in claim 9, wherein the mammal is preferably human.
